SEQUENCE LISTING (10 sequences)

SEQ ID NO: 1 - protein, 3 aa, Bos taurus
TVY

SEQ ID NO: 2 - protein, 8 aa, Bos taurus
NMAINPSK

SEQ ID NO: 3 - protein, 6 aa, Bos taurus
FALPQY

SEQ ID NO: 4 - protein, 7 aa, Bos taurus
FPQYLQY

SEQ ID NO: 5 - protein, 8 aa, Bos taurus
FALPQYLK

SEQ ID NO: 6 - protein, 6 aa, Bos taurus
NMAINP

SEQ ID NO: 7 - protein, 4 aa, Bos taurus
FALP

SEQ ID NO: 8 - protein, 11 aa, Bos taurus
ALNEINQFYQK

SEQ ID NO: 9 - protein, 9 aa, Bos taurus
ALNEINQFY

SEQ ID NO: 10 - protein, 2 aa, Bos taurus
YL

(19) United States
(12) Patent Application Publication
(10) Pub. No.: US 2006/0234942 A1
(43) Pub. Date: Oct. 19, 2006

(54) Use of at least one $g(a)62 casein peptide with angiotensin i converting enzyme inhibiting activity for preparing medicines, food products and food complements

(76) Inventors: Jerome Tauzin, Bauvin (FR); Laurent Miclo, Villers Les Nancy (FR); Catherine Lefranc, Villeneuve D'Ascq (FR); Jean-Francois Boudier, Agny (FR); Jean-Luc Gaillard, Luc Sur Mer (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/519,164

(22) PCT Filed: Jun. 24, 2003
(86) PCT No.: PCT/FR03/01945

§ 371 Date: Aug. 30, 2005

(30) Foreign Application Priority Data
Jun. 27, 2002 (FR) 02/08036

(51) Int. Cl. A61K 38/10; A61K 38/08; A61K 47/00

(52) U.S. Cl. 514/16; 514/17; 514/18; 424/439

(57) ABSTRACT

The invention relates to the use in the preparation of medicines having activity of the antihypertensive type, useful for treating or preventing hypertension, of one or more peptides having inhibiting activity on ACE with IC₅₀ values of the order of or less than 60 μM, selected from the group of peptides having the following amino acid sequences:

| | |
|---|---|
| Thr-Val-Tyr | (SEQ ID No.: 1) |
| Asn-Met-Ala-Ile-Asn-Pro-Ser-Lys | (SEQ ID No.: 2) |
| Phe-Ala-Leu-Pro-Gln-Tyr | (SEQ ID No.: 3) |
| Phe-Pro-Gln-Tyr-Leu-Gln-Tyr | (SEQ ID No.: 4) |
| Phe-Ala-Leu-Pro-Gln-Tyr-Leu-Lys | (SEQ ID No.: 5) |
| Asn-Met-Ala-Ile-Asn-Pro | (SEQ ID No.: 6) |
| Phe-Ala-Leu-Pro. | (SEQ ID No.: 7) |

The invention also relates to a pharmaceutical composition containing as its active principle an effective quantity of one or more of the above-cited peptides in combination with a pharmaceutically acceptable vehicle. The invention also relates to a food product, in particular suitable for supplementing the diet of people subject to hypertension or seeking to prevent the appearance thereof, containing an effective quantity of one or more of the above-cited peptides, in combination with food supports, in particular proteins, lipids, or carbohydrates. Such a food product may also include at least one of the peptides having the following amino acid sequences:

| | |
|---|---|
| Ala-Leu-Asn-Glu-Ile-Asn-Gln-Phe- | (SEQ ID No.: 8) |
| Tyr-Gln-Lys | |
| Ala-Leu-Asn-Glu-Ile-Asn-Gln-Phe- | (SEQ ID No.: 9) |
| Tyr | |
| Tyr-Leu. | (SEQ ID No.: 10) |

The present invention relates to the use of one or more peptides of bovine α_{S2} casein with inhibiting activity on angiotensin I converting enzyme in the preparation of medicines, food products, and food supplements having antihypertensive type activity.

### BACKGROUND OF THE INVENTION

Whole casein is a set of milk proteins that have been studied in depth, e.g. by Grosclaude (1), Swaisgood (2), and Grappin and Ribadeau-Dumas (3). Chromatography on diethylaminoethyl-cellulose (DEAE-cellulose) makes it possible to separate out from whole casein the following caseins: γ, κ, β, α_{S1} and α_{S2}. The amino acid sequences for caseins are well known [Eigel et al. (4), Holt and Sawyer (5)]; in particular the sequence for α_{S2} casein has been determined by Brignon et al. (6) and Stewart et al. (7).

It is already known that certain peptide fragments of these various caseins have a variety of biological activities [Clare and Swaisgood (8), Meisel (9)]. Concerning α_{S2} casein, the peptides CNα_{S2}-(f165-203) [Zucht et al. (10)], CNα_{S2}-(f183-107), and CNα_{S2}-(f164-179) [Recio and Visser (11)] presents antibacterial activity and the peptides CNα_{S2}-(f189-193), CNα_{S2}-(f190-197), and CNα_{S2}-(f198-202) inhibit angiotensin I converting enzyme [Corvol et al. (12)] with values for IC₅₀, i.e. the quantity of peptide needed for inhibiting 50% of the enzyme activity, equal to 580 μM, 300 μM, and 400 μM respectively [Maneo et al. (13)]. Nevertheless, those peptides do not present any significant antihypertensive effect in vivo in lines of rats that are spontaneously hypertensive, 6 hours after oral administration of a 1 milligram (mg) dose of synthesized peptide per kilogram (kg) of rat [Maneo et al. (13)].

Angiotensin I converting enzyme, referred to below as ACE, has a key role in vivo in regulating arterial pressure [Weber (14)]. ACE inhibitors (captoproil, benazepril, enalapril, lisinopril . . . ) [Pipeho (15)] are one of the main classes of molecules used for combating hypertension. They are particularly appropriate for diabetic patients and for heart or kidney failure [WHO (16), J.N.C. (17)].

According to the Applicant, it is important to make ACE inhibitors available that present IC₅₀ values that are well below those of the three above-mentioned peptides of α_{S2} casein. Values are taken as being “well below” when they are of the order of or less than 60 μM, it nevertheless being understood that a certain amount of inaccuracy remains concerning the value that is obtained as a function of operational conditions, so it is appropriate to refer to the conditions described below when determining said value.

The Applicant has found in in vitro tests that certain peptides of α_{S2} casein present inhibiting activity on ACE that has not previously been mentioned, with values of the order of less than 60 μM. These peptides are the following five peptides which can be obtained by trypsic hydrolysis of α_{S2} casein, namely: CNα_{S2}-(f25-32), CNα_{S2}-(f92-98), CNα_{S2}-(f174-179), CNα_{S2}-(f174-181), CNα_{S2}-(f182-184), and two other peptides obtained by chemical synthesis, namely CNα_{S2}-(f25-30) and CNα_{S2}-(f174-177).

### OBJECTS AND SUMMARY OF THE INVENTION

The present invention thus provides the use, in the preparation of medicines of the medicines having activity of the antihypertensive type, useful for treating or preventing hypertension, of one or more peptides having inhibiting activity on ACE with IC₅₀ values of the order of or less than 60 millimolar (μM), selected from the group of peptides having the following amino acid sequences:

Thr-Val-Tyr,
1
Asn-Met-Ala-Ile-Asn-Pro-Ser-Lys,
1 5
Phe-Ala-Leu-Pro-Gln-Tyr,
1 5
Phe-Pro-Gln-Tyr-Leu-Gln-Tyr,
1 5
Phe-Ala-Leu-Pro-Gln-Tyr-Leu-Lys,
1 5
Asn-Met-Ala-Ile-Asn-Pro,
1 5
Phe-Ala-Leu-Pro.
1

The present invention also provides pharmaceutical compositions containing as an active ingredient an effective quantity of at least one of said peptides in combination with a pharmaceutically acceptable vehicle.

The present invention also provides food products containing as an active principle at least one of said peptides, or else a total trypsic hydrolysate containing at least one of said peptides, or a fraction of said hydrolysate containing at least one of said peptides in combination with food supports, in particular proteins, lipids, or carbohydrates. Such food supplements can be suitable for supplementing the diets of people subject in particular to hypertension, or in order to prevent it appearing.

In the group of peptides of the present invention:

the peptide Thr-Val-Tyr, [TVY (SEQ ID No. 1)] of molecular weight 381.4, corresponds to the 182-184 peptide of α_{S2} casein;

the peptide Asn-Met-Ala-Ile-Asn-Pro-Ser-Lys, [NMAINPSK (SEQ ID No. 2)] of molecular weight 847.0 corresponds to the 25-32 peptide of α_{S2} casein;

the peptide Phe-Ala-Leu-Pro-Gln-Try, [FALPQY (SEQ ID No. 3)] of molecular weight 737.9 corresponds to the 174-179 peptide of α_{S2} casein;

the peptide Phe-Pro-Gln-Tyr-Leu-Gln-Tyr, [FPQYLQY (SEQ ID No. 4)] of molecular weight 958.1 corresponds to the 92-98 peptide of α_{S2} casein;

the peptide Phe-Ala-Leu-Pro-Gln-Tyr-Leu-Lys, [FALPQYLK (SEQ ID No. 5)] of molecular weight 979.2 corresponds to the 174-181 peptide of α_{S2} casein;

the peptide Asn-Met-Ala-Ile-Asn-Pro, [NMAINP (SEQ IS No. 6)] of molecular weight 658.8 corresponds to the 25-30 peptide of α_{S2} casein;

the peptide Phe-Ala-Leu-Pro, [FALP (SEQ ID No. 7)] of molecular weight 446.6 corresponds to the 174-177 peptide of α_{S2} casein.

Some of these peptides can be obtained from α_{S2} casein by enzymatic hydrolysis, preferably with the help of trypsin. They can then be concentrated or isolated by high performance liquid chromatography (HPLC) in reverse phase or using other chromatographic techniques (gel filtering, ion exchange, etc.), by centrifuging (on a membrane), or using other membrane separation techniques (micro filtration, ultrafiltration, etc. . . . ).

These peptides can also be obtained by chemical synthesis using methods that are well known to the person skilled in the art, such as those described, for example, by Merrifield (18).

Whole casein is obtained from milk by acid precipitation and by neutralization using an alkali in methods that are well known. For example, it is preferable to use the method of Nitschmann and Lehmann (19).

α_{S2} casein used as a starting material for obtaining peptides in the group selected in the context of the present invention can be obtained by conventional methods well known to the person skilled in the art starting from milk, whole casein, caseinates, and total protein concentrates of milk, e.g. obtained using the method described by Thomson (20) and Maubois (21).

For example, it is possible to prepare α_{S2} casein by adapting the method described by Sanogo et al. (22). That method is a method of fractioning on DEAE-cellulose using a discontinuous gradient of calcium chloride as the eluant. It enables all of the caseins to be fractioned quickly. It can advantageously be implemented using the DEAE-cellulose DE 23 [sold by Whatman, Maidstone, UK], which is a dry resin, as the anion exchanger support. After this step, in order to eliminate all traces of other proteins, an additional step of hydrophobic interaction chromatography may be performed applying a decreasing gradient of sodium phosphate to the TSKgel phenyl 5PW column [TosoHaas, Stuttgart, Germany].

The total trypsic hydrolysate of α_{S2} casein is obtained by the action of trypsin on α_{S2} casein, e.g. under the conditions described below.

The first, second, third, fourth, and fifth peptides [SEQ ID No.: 1, 2, 3, 4, 5] of the group selected in the context of the present invention are purified directly from the total trypsic hydrolysate by reverse phase HPLC using a gradient of acetonitrile. Each of the collected peptide peaks corresponding to these five peptides are lyophylized.

Each of these five peptides, alone or in a mixture, or a fraction of the total trypsic hydrolysate containing at least one of these five peptides, or the total trypsic hydrolysate containing all five peptides, can be used as an active principle either in food supplements in combination with food supports (e.g. proteins, lipids, or carbohydrates), or in food products for a particular diet.

The medicines useful in treating hypertension prepared using at least one of the seven peptides of the group selected in the context of the present invention can be administered orally.

For oral administration, pharmaceutical compositions need to be in the form of pills, capsules, powders, granules, or any other form suitable for oral administration.

### DETAILED DESCRIPTION OF THE INVENTION

The invention is described below in greater detail by way of the following non-limiting example:

A—Preparing α_{S2} Casein

Five grams (g) of ammonium caseinate were dissolved in 200 milliliters (mL) of 20 mM acetate buffer having a pH of 6.6, and containing 3.5 M of urea, 35 mM of ethylenediaminetetraacetic acid (EDTA), and 0.1% of 2-mercaptoethanol, and then 20 g of DEAE-cellulose DE 23 balanced in 150 mL of the same buffer were added. The resulting mixture was stirred for 15 minutes (min) at 25° C. and then filtered on a No. 41 filter [Whatman]. The retentate was eluted with twice 250 mL of acetate-urea-EDTA buffer in 2-mercaptoethanol. The three filtrates were grouped together. This first stirring-filtering cycle served to eliminate a fraction F0. The following casein fractions (F1 and F2) were eluted using the same procedure, adding 30 mM and 70 mM of CaCl₂ respectively to the buffer. EDTA was added to the fractions in amounts of 15 mM to the fraction F1, 45 mM to the fraction F1, and 85 mM to the fraction F2. The filtrates F0, F1, and F2, were dialyzed against ultrapure water and then lyophylized, after which they were subjected to electrophoresis using a polyacrylamide-urea gel in order to reveal the fractioning. The fraction F1 contained α_{S2} casein.

The purification of the α_{S2} casein was finished off by hydrophobic interaction chromatography on a TSKgel phenyl 5PW column [TosoHaas, Stuttgart, Germany] having dimensions of 150 millimeters (mm)×32.5 mm. The fraction F1 (1 milligram per milliliter (mg·mL⁻¹)) was put into solution in a 0.48 M sodium phosphate buffer at pH 6.4, containing 2.5 M of urea and in the presence of 0.1% 2-mercaptoethanol, and then filtered on a 0.45 micrometer (μm) PVDF filter [Pall Corporation, Ann Arbor, Mich., United States]. Twenty mg of protein solution were injected. A non-linear gradient going from 0.48 M to 0.037 M of sodium phosphate heaving a pH of 6.4 and containing 2.5 M of urea was applied at a flow rate of 6.0 milliliters per minute (mL·min⁻¹) as follows: from 480 mM to 126 mM (18 min), 126 mM (3 min), from 126 mM to 103 mM (3 min), 103 mM (3 min), from 103 mM to 72 mM (5 min), 72 mM (5 min), from 72 mM to 37 mM (4 min), 37 mM (17 min). The collected bovine α_{S2} casein was dialyzed, lyophylized, and stored under a vacuum at +4° C.

B—Preparing the Trypsic Hydrolysate of α_{S2} Casein

The α_{S2} casein was put into solution at a concentration of 0.05% (w/v) in 100 mL of 67 mM sodium phosphate buffer at a pH of 8.1 containing 0.02% sodium nitride. Bovine pancreatic trypsin (E.C. 3.4.21.4) immobilized on agarose beads and treated by TPCK (N-tosyl-L-phenylalanine chloromethylketone) [Sigman, Saint Louis, Miss., United States] was added, after washing in the preceding buffer and filtering several times, to the α_{S2} casein solution in order to obtain a concentration of 0.2 units of Nα-benzoyl-L-arginine ethyl ester (BAEE) per mL. Hydrolysis took place at 37° C. for 24 hours. The reaction was stopped by diluting the mixture twice using 4% acetonitrile containing 0.2% trifluoroacetic acid (TFA), and then filtering on a 0.45 μm polyvinylidene fluoride (PVDF) filter. The hydrolysate was conserved at −30° C.

C—Fractioning the Hydrolysate by Reverse-Phase HPLC in a Gradient of Acetonitrile

The hydrolysate was fractioned on a C18 XTerra™ column [Waters, Milford, Mass., United States] having dimensions of 250 mm×4.6 mm thermostated to 37° C. 500 μL of sample (0.25 mg·mL⁻¹) were injected. The elution profile had an isocratic phase of 3 min at 1.6% acetonitrile in water (in the presence of 0.1% TFA) followed by a linear gradient serving to reach 40% acetonitrile in 87 min at a rate of 1 mL·min⁻¹.

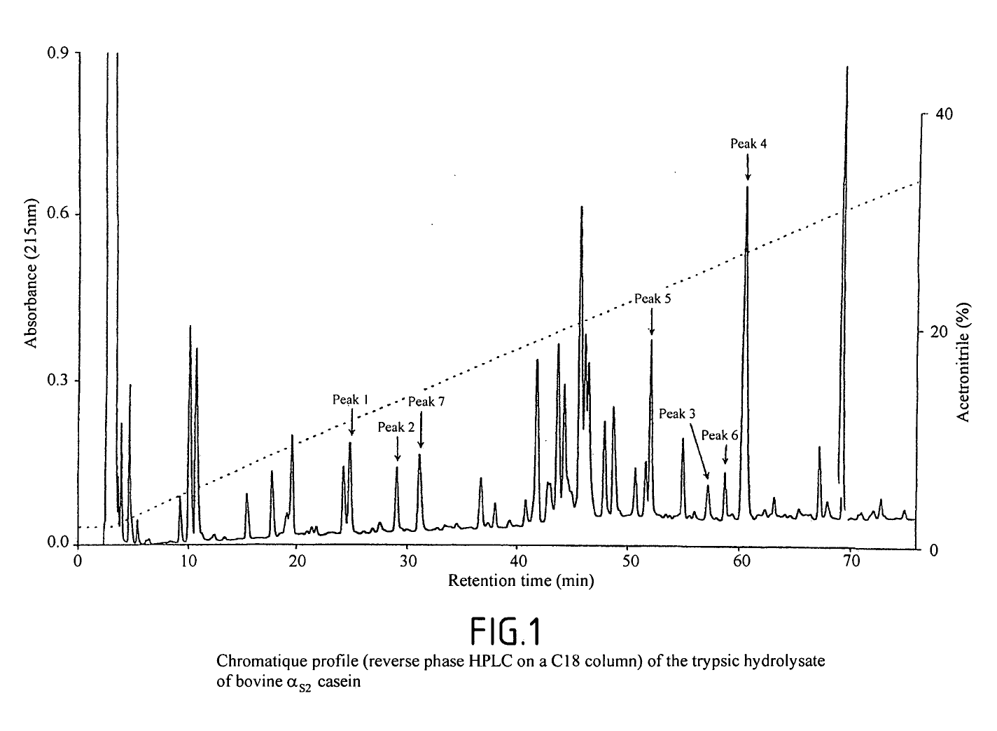

The peptide profile is shown in FIG. 1 where the absorbance at 215 nanometers (nm) is plotted up the ordinate and elution time along the abscissa.

Five of the seven peptides of the group selected in the context of the present invention correspond to the peptide peaks referenced 1 to 4 in FIG. 1. These peptides were selected and lyophylized twice. They were identified by determining their amino acid composition by the Hamilton (23) ninhydrine method and by mass spectrometry coupled to the HPLC, ESI-LC/MS (“electrospray source ionization”), or by MS/MS, mass spectrometry in tandem.

The peak 1 collected at 25 min contains the peptide TVY (SEQ ID No.: 1).

The peak 2 collected at 29 min contains the peptide NMAINPSK (SEQ ID No.: 2).

The peak 3 collected at 57 min contains the peptide FALPQY ID No.: 3).

The peak 4 collected at 60 min contains the peptides FPQYLQY (SEQ ID No. 4) and FALPQYLK (SEQ ID No.: 5).

The other two peptides, NMAINP (SEQ ID No.: 6) and FALP (SEQ ID No.: 7) can be obtained by chemical synthesis using conventional methods. The same applies to the five peptides that are preferably obtained by fractioning the total trypsic hydrolysate of α_{S2} casein.

D—In Vitro Test of the Peptides on the Enzyme for the Angiotensin I Converting Enzyme (ACE)

The main experiment relies on measuring the residual activity of ACE on a synthesized substrate of Hippurhyl-His-Leu-OH in the presence of a potentially inhibiting peptide [Cushman and Cheung (24)]. The hippuric acid that was released was assayed by HPLC and its quantity compared with a reference having no inhibitor.

Incubation was performed in a 50 mM CHES buffer with a pH of 8.3 containing 5 mM of hippuryl-His-Leu-OH, 350 mM of NaCl, 3.33 U·L⁻¹ ACE, and 5% ethanol. The mixture (final volume: 150 μL), after 10 min of pre-incubation without the enzyme, was incubated for 60 min at 37° C. The reaction was stopped with captopril (5 μM), EDTA (1 mM), and TFA (0.067%). The hippuric acid that was released was quantified by HPLC using a C18 Symmetry® column [Waters, Milford, Mass., United States] with dimensions of 150 mm×2.1 mm and thermostated at 37° C. The samples were filtered on a 0.45 μm PVDF filter and 40 μL were injected. An acetonitrile gradient in water (in the presence of 0.1% TFA) was applied at a rate of 0.25 mL·min⁻¹. The elution gradient went from 13% to 50% acetonitrile in 7 min, and then reached 99% in 0.5 min, and was maintained at that value for 1.5 min.

The method of determining the IC₅₀ was validated by comparing the value found for captopril (0.022 μm), a known ACE inhibitor, with biological values (0.023 μm [Cushman et al. (25)], 0.018 μm [Duncan et al. (26)], 0.007 μm [Pihlanto-Leppälä et al. (27)].

The four chromatographic peaks (1 to 4) collected from the trypsic hydrolysate of α_{S2} casein and corresponding to the five peptides of the group selected in the context of the present invention were tested twice at a concentration of 50 μM of primary amines. The chromatographic peaks numbered 5 to 7 were tested under the same conditions.

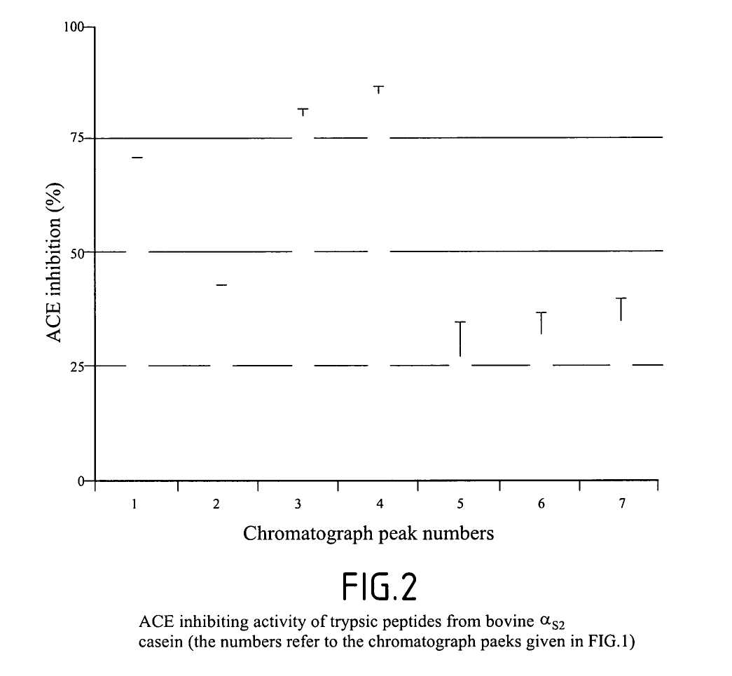

The results obtained are given in FIG. 2 where the inhibition percentage is plotted up the ordinate and the chromatographic peak number along the abscissa. It can be seen that the peaks 1 to 4 containing the peptides of the group selected in the context of the present invention inhibits ACE at more than 40%, and of those peaks, peak No. 4 containing the peptides FPQYLQY (SEQ ID No. 4) and FALPQYLK (SEQ ID No. 5), peak No. 3 containing the peptide FALPQY (SEQ ID No. 3), and peak No. 1 containing the peptide TVY (SEQ ID No. 1) inhibit ACE at more than 70%.

Synthetic peptides were used to determine the IC₅₀ values of these five peptides precisely. The peptides were initially tested twice at concentrations lying in the range 0.1 μM and 250 μM to 500 μM in order to obtain an estimate of their IC₅₀ value, and then tested in triplicate on an appropriate range of concentrations.

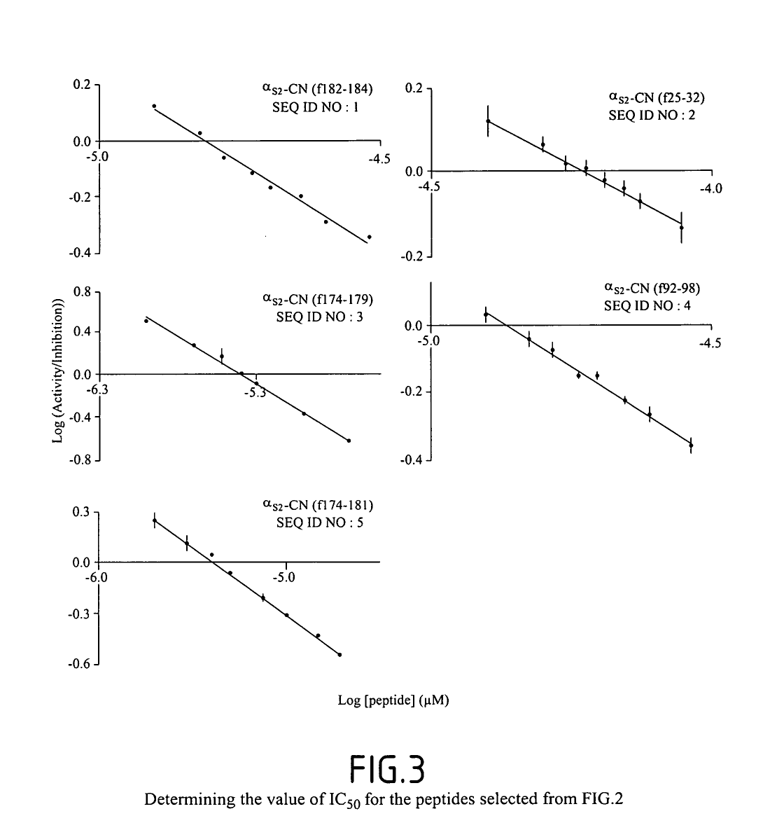

The results obtained are given by the graphs of FIG. 3 where the logarithm of the activity/inhibition ratio is plotted up the ordinate and the logarithm of peptide concentration along the abscissa. This enables the inhibition curve to be liberalized and enables the IC₅₀ values to be deduced therefrom using straight line equations. The IC₅₀ values are summarized in Table 1.

They are all of the order of or less than 60 μM, it being observed that the peptides FALPQY (SEQ ID No. 3) and FALPQYLK (SEQ ID No. 5) have the best performance with an IC₅₀ value of 4.3 μM.

The seven peptides in the group selected in the context of the present invention have amino acid sequences that are different from those of the eight inhibitor peptides described in the past [Fitzgerald and Meisel (28), Yamamoto and Takano (29), Pihlanto-Leppälä (30), Nurminen (31), Takano (32)], including those reported by Maeno et al. (13) obtained using α_{S2} casein: CNα_{S2}-(f198-202), CNα_{S2}-(f190-197), and CNα_{S2}-(f189-193). As mentioned above, two peptides of the group selected in the context of the present invention, obtained by fractioning the trypsic hydrolysate of α_{S2} casein gave values of IC₅₀ of less than 5 μM, and two others gave values for IC₅₀ less than 20 μM, thereby classifying them amongst the most active inhibitors of ACE amongst natural peptides obtained by a mono-enzymatic process on milk proteins.

The two peptides NMAINP (SEQ ID No. 6) and FALP (SEQ ID No. 7) which are not obtained directly by fractioning the trypsic hydrolysate of α_{S2} casein are remarkable firstly in that they possess a prolyl residue at their C-terminal end, which is common with certain other ACE-inhibiting peptides [Maruyama, et al. (33), Kohmura et al. (34, 35, 36), Nakamura et al. (37)], and secondly in that their amino acid sequence is completely contained in the other two peptides NMAINPSK (SEQ ID No. 2) and FALPQY (SEQ ID No. 3) which are obtained directly by such fractioning. As a result, it is possible to envisage that the use of the second two peptides (SEQ ID Nos. 2 and 3) as medicine, or as a food supplement, could lead to in vivo formation of the first two peptides (SEQ ID Nos. 6 and 7) by breaking the appropriate peptide bonds.

It should be observed that using at least one of the seven peptides of the group selected in the context of the present invention for preparing medicines, food products, or food supplements may be performed in combination with one or more other peptides, having ACE-inhibiting activity but having an IC₅₀ value greater than 60 μM. This would occur when implementing total trypsic hydrolysate of α_{S2} casein or a fraction thereof containing at least one peptide of the group. Such a combination could be advantageous for in vivo inhibiting activity on ACE.

This combination preferably makes use of the following peptides:

(SEQ ID No. 8), CNα_{S2}-(f81-91), ALNEINQFYQK, Ala-Leu-Asn-Glu-Ile-Asn-Gln-Phe-Try-Gln-Lys, peak 5 eluted at 52 min;

(SEQ ID No. 9), CNα_{S2}-(f81-89), ALNEINQFY, Ala-Leu-Asn-Glu-Ile-Asn-Gln-Phe-Tyr, peak 6 eluted at 59 min;

(SEQ ID No. 10), CNα_{S2}-(f206-207), YL, Tyr-Leu, peak 7 eluted at 31 min;

which may also be obtained by fractioning the trypsic hydrolysate of α_{S2} casein and which inhibits ACE in the range 25% to 35% at a concentration of 50 μM of primary amines (FIG. 2).

### BIBLIOGRAPHIC REFERENCES

(1) GROSCLAUDE, F., 1988, Le polymorphisme des principales lactoprotéines bovines, INRA Prod. Anim., 1, 5-17.
(2) SWAISGOOD, H. E., 1992, Chemistry of the caseins in P. F. Fox: Advanced dairy chemistry, volume 1, Proteins, Blackie Academic & Professional, London, United Kingdom, 63-109.
(3) GRAPPIN, R. and RIBADEAU-DUMAS, B., 1992, Analytical methods for milk proteins in P. F. Fox: Advanced dairy chemistry, volume 1, Proteins, Blackie Academic & Professional, London, United Kingdom, 1-61.
(4) EIGEL, W. N., BUTLER, J. E., ERNSTROM, C. A., FARRELL, H. M., HARWALKAR, V. R., JENNESS, R. and WHITNEY, R. McL., 1984, Nomenclature of proteins of cow's milk : fifth revision, J. Dairy Sci., 67, 1599-1631.
(5) HOLT, C. and SAWYER, L., 1988, Primary and predicted secondary structures of the caseins in relation to their biological functions, Protein Eng., 2, 251-259.
(6) BRIGNON, G., RIBADEAU-DUMAS, B., MERCIER, J.-C., PELISSIER, J.-P. and DAS, B. C., 1977, Complete amino acid sequence of bovine α_{S2}-casein, FEBS Lett., 76, 274-279.
(7) STEWART, A. F., BONSING, J., BEATTIE, C. W., SHAH, F., WILLIS, I. M. and MACKINLAY, A. G., 1987, Complete nucleotide sequence of bovine α_{S2} and β-casein cDNAs: comparisons with related sequences in other species, Mol. Biol. Evol., 4, 231-241.
(8) CLARE, D. A. and SWAISGOOD, H. E., 2000, Bioactive milk peptides: a prospectus, J. Dairy Sci., 83, 1187-1195.
(9) MEISEL, H., 1997, Biochemical properties of regulatory peptides derived from milk proteins, Biopolymers, 43, 119-128.
(10) ZUCHT, H.-D., RAIDA, M., ADERMANN, K, MÄGERT, H.-J. and FORSSMANN, W.-G., 1995, Casocidin-I: a casein-α_{S2} derived peptide exhibits antibacterial activity, FEBS Lett., 372, 185-188.
(11) RECIO, I. and VISSER, S., 1999, Identification of two distinct antibacterial domains within the sequence of bovine-αₛ₂, Biochim. Biophys. Acta, 1428, 314-326.
(12) CORVOL, P., WILLIAMS, T. A., SOUBRIER, F., 1995, Peptidyl dipeptidase A: angiotensin I-converting enzyme, Methods Enzymol., 248, 243-305.
(13) MAENO, M., YAMAMOTO, N. and TAKANO, T., 1996, Identification of an antihypertensive peptide from casein hydrolysate produced by a proteinase from Lactobacillus helveticus CP790, J. Dairy Sci., 79, 1316-1321.
(14) WEBER, M. A., 1999, Interrupting the renin-angiotensin system: the role of angiotensin-converting enzyme inhibitors and angiotensin II receptor antagonists in the treatment of hypertension, 12, 189S-194S.
(15) PIEPHO, R. W., 2000, Overview of the angiotensin-converting-enzyme inhibitors, Am. J. Health-Syst. Pharm., 57, S3-S7.
(16) Guidelines subcommittee, 1999, World Health Organization-International Society of Hypertension. Guidelines for the management of hypertension, J. Hypertens., 17, 151-183.
(17) Joint National Committee, 1997, Detection and treatment of high blood pressure. The sixth report of the joint national committee on prevention and treatment of high blood pressure (JNC VI), Arch. Intern. Med., 157, 2413-2446.
(18) MERRIFIELD, R. B., 1963, Solid phase peptide synthesis I. Synthesis of a tetrapeptide, J. Amer. Chem. Soc., 85, 2149-2154.
(19) NITSCHMANN, H. S. and LEHMANN, W., 1947, Zum problem der labwirkung auf casein, Helv. Chim. Acta, 130, 804.
(20) THOMSON, A. R., 1984, Recent developments in protein recovery and purification, J. Chem. Tech. Biotechnol., 34B, 190-198.
(21) MAUBOIS, J.-L., 1984, Separation, extraction and purification of milk protein components, Lait, 64, 485-495.
(22) SANOGO, T., PAQUET, D., AUBERT, F. and LINDEN, G., 1989. Purification of α_{S1}-casein by fast protein liquid chromatography, J. Dairy Sci., 72, 2242-2246.
(23) HAMILTON, P. B., 1963, Ion exchange chromatography of amino acids. A single column, high resolving, fully automatic procedure, Anal. Chem., 35, 2055-2063.
(24) CUSHMAN, D. W. and CHEUNG, H. S., 1971, Spectrophotometric assay and properties of the angiotensin-converting enzyme of rabbit lung, Biochem. Pharm., 20, 1637-1648.
(25) CUSHMAN, D. W., CHEUNG, H. S., SABO, E. F. and ONDETTI, M. A., 1977, Design of potent competitive inhibitors of angiotensin-converting enzyme. Carboxyalkanoyl and mercaptoalkanoyl amino acids, Biochemistry, 16, 5484-5491.
(26) DUNCAN, A. C., JÄGER, A. K. and VAN STADEN, J., 1999, Screening of Zulu medicinal plants for angiotensin converting enzyme (ACE) inhibitors, J. Ethnopharm., 68, 63-70.
(27) PIHLANTO-LEPPÄLÄ, A., ROKKA, T. and KORHONEN, H., 1998, Angiotensin I converting enzyme inhibitory peptides derived from bovine milk proteins, Int. Dairy J., 8, 325-331.
(28) FITZGERALD, R. J. and MEISEL, H., 2000, Milk protein-derived inhibitors of angiotensin-I-converting enzyme, British J. Nutr., 84, S33-S37.
(29) YAMAMOTO, N. and TAKANO, T., 1999, Antihypertensive peptides derived from milk proteins, Nahrung, 3, S159-S164.
(30) PIHLANTO-LEPPÄLÄ, A., 2001, Bioactive peptides derived from bovine whey proteins: opioid and ace-inhibitory peptides. Trends Food Sci. Tech., 11, 347-356.
(31) NURMINEN, M.-L., 2000, Milk-derived peptides and blood pressure, Bull. IDF, 353, 11-15.
(32) TAKANO, T., 1998, Milk derived peptides and hypertension-reduction, Int. Dairy J., 8, 375-381.
(33) MARUYAMA, S., NAKAGOMI, K., TOMIZUKA, N. and SUZUKI, H., 1985, Angiotensin I-converting enzyme inhibitor derived from an enzymatic hydrolysate of casein. II. Isolation and bradykinin-potentiating activity on the uterus and the ileum of rats, Agric. Biol. Chem., 49, 1404-1409.
(34) KOHMURA, M., NIO, N., KUBO, K. MINOSHIMA, Y., MUNEKATA, E. and ARIYOSHI, Y., 1989, Inhibition of angiotensin-converting enzyme by synthetic peptides of human β-casein, Agric. Biol. Chem., 53, 2107-2114.
(35) KOHMURA, M., NIO, N. and ARIYOSHI, Y., 1990a, Inhibition of angiotensin-converting enzyme by synthetic fragments of human κ-casein, Agric. Biol. Chem., 54, 835-836.
(36) KOHMURA, M., NIO, N. and ARIYOSHI, Y., 1990b, Inhibition of angiotensin-converting enzyme by synthetic peptide fragments of various β-casein, Agric. Biol. Chem., 54, 1101-1102.

(37) NAKAMURA, Y., YAMAMOTO, N., SAKAI, K. and TAKANO, T., 1995, Antihypertensive effect of sour milk and peptides isolated from it that are inhibitors to angiotensin I converting enzyme, J. Dairy Sci., 78, 1253.

TABLE 1

| | | | | | |
|---|---|---|---|---|---|
| | | | ID | Inhibition | IC₅₀ |
| Inhibitor | No.^{a} | Sequence | No.^{b} | (%)^{c} | (μM) |

| | | | | | |
|---|---|---|---|---|---|
| Captopril | | | | >99.5 | 0.022 |
| CNα_{S2}- | 1 | TVY | 1 | 70.2 | 15 |
| (f182-184) | | | | | |
| CNα_{S2}- | 2 | NMAINPSK | 2 | 42.5 | 60 |
| (f25-32) | | | | | |
| CNα_{S2}- | 3 | FALPQY | 3 | 82.7 | 4.3 |
| (f174-179) | | | | | |
| CNα_{S2}- | 4 | FPQYLQY | 4 | 86.0^{d} | 14 |
| (f92-98) | | | | | |
| CNα_{S2}- | 4 | FALPQYLK | 5 | 86.0^{d} | 4.3 |
| (f174-181) | | | | | |
| CNα_{S2}- | 5 | ALNEINQFYQK | 8 | 27.2 | 26.4 |
| (f81-91) | | | | | |
| CNα_{S2}- | 6 | ALNEINQFY | 9 | 32.2 | 219 |
| (f81-89) | | | | | |
| CNα_{S2}- | 7 | YL | 10 | 34.8 | nd |
| (f206-207) | | | | | |

^{a}Peak number in HPLC of FIG. 1.
^{b}Peptide sequence ID number.
^{c}Determined with a primary amine or captopril concentration equal to 50 μM.
^{d}CNα_{S2}-(f92-98) and CNα_{S2}-(f174-181) being mixed together in peak No. 4.
nd Not determined.

What is claimed is:

1. The use for the preparation of medicines having activity of the antihypertensive type, useful for treating or preventing hypertension, of one or more peptides having inhibiting activity on ACE with IC₅₀ values of the order of or less than 60 μM, selected from the group of peptides having the following amino acid sequences:

| | |
|---|---|
| Thr-Val-Tyr | (SEQ ID No.: 1) |
| Asn-Met-Ala-Ile-Asn-Pro-Ser-Lys | (SEQ ID No.: 2) |
| Phe-Ala-Leu-Pro-Gln-Tyr | (SEQ ID No.: 3) |
| Phe-Pro-Gln-Tyr-Leu-Gln-Tyr | (SEQ ID No.: 4) |
| Phe-Ala-Leu-Pro-Gln-Tyr-Leu-Lys | (SEQ ID No.: 5) |
| Asn-Met-Ala-Ile-Asn-Pro | (SEQ ID No.: 6) |
| Phe-Ala-Leu-Pro. | (SEQ ID No.: 7) |

2. A pharmaceutical composition containing as its active principle an effective quantity of one of more peptides having inhibiting activity on ACE, with IC₅₀ values of the order of or less than 60 μM, selected from the group of peptides having the following amino acid sequences:

| | |
|---|---|
| Thr-Val-Tyr | (SEQ ID No.: 1) |
| Asn-Met-Ala-Ile-Asn-Pro-Ser-Lys | (SEQ ID No.: 2) |
| Phe-Ala-Leu-Pro-Gln-Tyr | (SEQ ID No.: 3) |
| Phe-Pro-Gln-Tyr-Leu-Gln-Tyr | (SEQ ID No.: 4) |
| Phe-Ala-Leu-Pro-Gln-Tyr-Leu-Lys | (SEQ ID No.: 5) |
| Asn-Met-Ala-Ile-Asn-Pro | (SEQ ID No.: 6) |
| Phe-Ala-Leu-Pro | (SEQ ID No.: 7) |

in combination with a pharmaceutically acceptable vehicle.

3. A food product, in particular one that is useful for supplementing the diet of people subject to hypertension or desiring to prevent the appearance thereof, the food product containing an effective quantity of one or more peptides having inhibiting activity on ACE, with IC₅₀ values of the order of or less than 60 μM, selected from the group of peptides having the following amino acid sequences:

| | |
|---|---|
| Thr-Val-Tyr | (SEQ ID No.: 1) |
| Asn-Met-Ala-Ile-Asn-Pro-Ser-Lys | (SEQ ID No.: 2) |
| Phe-Ala-Leu-Pro-Gln-Tyr | (SEQ ID No.: 3) |
| Phe-Pro-Gln-Tyr-Leu-Gln-Tyr | (SEQ ID No.: 4) |
| Phe-Ala-Leu-Pro-Gln-Tyr-Leu-Lys | (SEQ ID No.: 5) |
| Asn-Met-Ala-Ile-Asn-Pro | (SEQ ID No.: 6) |
| Phe-Ala-Leu-Pro | (SEQ ID No.: 7) |

in combination with food supports, in particular proteins, lipids, or carbohydrates.

4. A food product according to claim 3, including a fraction of trypsic hydrolysate of α_{S2} casein containing at least one of the peptides having the following amino acid sequences:

| | |
|---|---|
| Thr-Val-Tyr | (SEQ ID No.: 1) |
| Asn-Met-Ala-Ile-Asn-Pro-Ser-Lys | (SEQ ID No.: 2) |
| Phe-Ala-Leu-Pro-Gln-Tyr | (SEQ ID No.: 3) |
| Phe-Pro-Gln-Tyr-Leu-Gln-Tyr | (SEQ ID No.: 4) |
| Phe-Ala-Leu-Pro-Gln-Tyr-Leu-Lys. | (SEQ ID No.: 5) |

5. A food product according to claim 3, including the total trypsic hydrolysate of α_{S2} casein, containing the five peptides having the following amino acid sequences:

| | |
|---|---|
| Thr-Val-Tyr | (SEQ ID No.: 1) |
| Asn-Met-Ala-Ile-Asn-Pro-Ser-Lys | (SEQ ID No.: 2) |
| Phe-Ala-Leu-Pro-Gln-Tyr | (SEQ ID No.: 3) |
| Phe-Pro-Gln-Tyr-Leu-Gln-Tyr | (SEQ ID No.: 4) |
| Phe-Ala-Leu-Pro-Gln-Tyr-Leu-Lys. | (SEQ ID No.: 5) |

6. A food product according to claim 3, including at least one of the peptides having the following amino acid sequences:

| | |
|---|---|
| Ala-Leu-Asn-Glu-Ile-Asn-Gln-Phe- | (SEQ ID No.: 8) |
| Tyr-Gln-Lys | |
| Ala-Leu-Asn-Glu-Ile-Asn-Gln-Phe- | (SEQ ID No.: 9) |
| Tyr | |
| Tyr-Leu. | (SEQ ID No.: 10) |
